(19) United States
(12) Patent Application Publication
(10) Pub. No.: US 2001/0002383 A1
(43) Pub. Date: May 31, 2001

(54) Molded catalysts

(76) Inventors: Toshio Hidaka, Tsukuba-shi (JP); Yasushi Miki, Tsukuba-shi (JP); Takeshi Kawai, Tsukuba-shi (JP)

Correspondence Address: Pillsbury Madison & Sutro LLp, Intellectual Property Group, 1100 New York Avenue, NW, Ninth Floor, East Tower, Washington, DC, 20005-3918, US

(21) Appl. No.: 09/725,013

(22) Filed: Nov. 29, 2000

(30) Foreign Application Priority Data
Nov. 29, 1999 (JP) 337777

(51) Int. Cl. C07C 209/00

(52) U.S. Cl. 502/72; 564/479

(57) ABSTRACT

A molded catalyst is provided, which is a crystalline aluminosilicate molecular sieve or crystalline silicoaluminophosphate molecular sieve, containing swelling synthetic mica, as binder. The present molded catalyst exhibits a sufficient disruptive strength even with a small amount of the binder added.

### BACKGROUND OF THE INVENTION

1. Field of the Invention

The present invention relates to molded catalysts, more particularly, to those having excellent fabrication and disruptive strength, obtained by using synthetic mica as a binder for crystalline molecular sieves. It is particularly significant in the field of industrial catalysts to obtain a molded catalyst having excellent fabrication and disruptive strength, with a small amount of binder.

2. Description of the Prior Art

Generally, solid catalysts are practically used after being shaped by tableting, extrusion molding, spray drying or other means. Molecular sieves, such as zeolite, have been shaped usually by extrusion molding. In this process, silica or alumina has been used as binders in an amount of 20% by weight or more, preferably about 50% by weight, in order to obtain a sufficient mechanical strength. However, the addition of any binders causes relative decrease in the catalytic activity. As a result, the amount of catalyst to be filled is reluctantly increased, in order to keep the desired catalytic activity. Accordingly, a binder is desirable, which yields a practically large mechanical strength to molded catalysts with an adding amount as small as possible. Such kind of binders, however, has not yet been available.

### SUMMARY OF THE INVENTION

An object of the invention is to provide a binder for catalyst molding, which yields a sufficient mechanical strength to molded catalysts even with a small adding amount.

After exhaustive investigations to solve such problems, the present inventors have accomplished the invention, based on the findings that the molded catalyst prepared from a molecular sieve, and having swelling synthetic mica blended, has an excellent mechanical strength, even with a small amount thereof.

The present invention relates to

(1) a molded catalyst comprising a crystalline alumino-silicate molecular sieve or crystalline silicoaluminophosphate molecular sieve, containing swelling synthetic mica, as binder;

(2) a molded catalyst according to (1), which contains silica, alumina, titania, zirconia, yttria, sericite, kaolinite or montmorillonite;

(3) a molded catalyst according to (1), wherein the crystalline aluminosilicate molecular sieve or crystalline silicoaluminophosphate molecular sieve contains Li, Na, Be, Mg, Ca, Sr, Y, Ti, Zr, V, Nb, Cr, Mn, Fe, Ru, Co, Rh, Ir, Ni, Pd, Pt, Cu, Zn, B, Ga, In, Ge or Sn;

(4) a molded catalyst according to (1), wherein the crystalline aluminosilicate molecular sieve is mordenite, chabazite, erionite, ferrierite, faujasite, levyne, ZSM-5, zeolite A, zeolite β, zeolite Y, FU-1, Rho, ZK-5, RUB-3, RUB-13, NU-3, NU-4, NU-5, NU-10, NU-13, NU-23 or MCM-22;

(5) a molded catalyst according to (1), wherein the crystalline silicoaluminophosphate molecular sieve is SAPO-5, SAPO-11, SAPO-17, SAPO-18, SAPO-26, SAPO-31, SAPO-33, SAPO-34, SAPO-35, SAPO-42, SAPO-43, SAPO-44, SAPO-47 or SAPO-56;

(6) a molded catalyst according to (1), wherein the crystalline molecular sieve is mordenite containing Ti, Y or Zr, or SAPO-34 containing Ti, Y or Zr, being used for producing methylamines;

(7) a process for producing methylamines wherein methanol and ammonia are allowed to react in the presence of the molded catalyst mentioned in (1); and

(8) a process for producing methylamines wherein monomethylamines is subjected to a disproportionation reaction in the presence of the molded catalyst mentioned in (1).

### DESCRIPTION OF THE PREFERRED EMBODIMENTS

Kind of the molecular sieves used in the present invention is not limitative, but preference is those having micropore diameters suitable for a desired reaction. In the production of methylamines through a reaction of methanol and ammonia, for example, a molecular sieve having micropore diameters ranging from 0.3 to 0.6 nm is preferable. According to the IUPAC structural codes, 8-membered ring-structural ABW, AEI, AFX, APC, ATN, ATT, ATV, AWW, CHA, DDR, EAB, ERI, GIS, JBW, KFI, LEV, LTA, MER, MON, PAU, PHI, RHO, RTE, RTH and VNI; 9-membered ring-structural CHI, LOV, RSN and VSV; 10-membered ring-structural DAC, EPI, FER, LAU, MEL, MFI, MFS, MTT, NES, TON and WEI; and 12-membered ring-structural AFS, AFY, ATO, CAN, GME, MAZ, MEI, MTW, OFF, -RON and VET may be used.

Among these known molecular sieves, crystalline alumino-silicate molecular sieves, such as mordenite, chabazite, erionite, ferrierite, faujasite, levyne, ZSM-5, zeolite A, zeolite β, zeolite Y, FU-1, Rho, ZK-5, RUB-3, RUB-13, NU-3, NU-4, NU-5, NU-10, NU-13, NU-23 and MCM-22; and crystalline silicoaluminophosphate molecular sieves, such as SAPO-5, SAPO-11, SAPO-17, SAPO-18, SAPO-26, SAPO-31, SAPO-33, SAPO-34, SAPO-35, SAPO-42, SAPO-43, SAPO-44, SAPO-47 and SAPO-56, are specifically illustrated as the present catalyst.

In the production of methylamines through a reaction of methanol and ammonia, as mentioned above, the particularly preferable crystalline molecular sieve includes mordenite, chabazite, erionite, ferrierite, levyne, faujasite, ZSM-5, zeolite A, zeolite β, zeolite Y, FU-1, Rho, ZK-5, RUB-3, RUB-13, NU-3, NU-4, NU-5, NU-10, NU-13, NU-23, MCM-22, SAPO-5, SAPO-11, SAPO-17, SAPO-18, SAPO-26, SAPO-31, SAPO-33, SAPO-34, SAPO-35, SAPO-42, SAPO-43, SAPO-44, SAPO-47 and SAPO-56. Mordenite and SAPO-34 are the most preferable. These molecular sieves may be used singly or as a mixture of suitably selected ones.

These crystalline molecular sieves are preferably of H-type. More preferably, the molecular sieve may contain a metal through substitution of a part of the H-type structure with Li, Na, Be, Mg, Ca, Sr, Y, Ti, Zr, V, Nb, Cr, Mn, Fe, Ru, Co, Rh, Ir, Ni, Pd, Pt, Cu, Zn, B, Ga, In, Ge or Sn. Alternatively, coating may be made with a compound containing such a metal. These steps improve the activity and selectivity in the reaction. Particularly, inclusion of Ti, Y, or Zr, as a metal element, or in the form of the oxide, is preferable.

As for the metal sources, water soluble salts of the metal, such as the nitrate, sulfate and chloride, are preferred. Such a metal may be added to a crystalline molecular sieve by impregnation or mechanical mixing of the salt, or by chemical deposition through thermal decomposition, or addition in advance to the material mixture used for a hydrothermal synthesis. The amount ratio of the metal in the molecular sieve is preferably 0.05 to 20% by weight.

Clay compounds, including kaolinite, selisite, talc, mica, montmorillonite, sepiolite, attapulgite and smectite, have generally been used as binder for catalyst molding. The mica referred to herein is a kind of hydrous aluminosilicate minerals, and includes muscovite, phlogopite, biotite, lepidolite, vandium mica, chromium mica, fluorine mica and the like. A feature of the invention is the use of swelling synthetic mica, preferably synthetic fluorine mica.

Swelling synthetic mica is prepared by a melting method or solid phase method, using talc, as the main material, to which fluorine and/or sodium sources are added. Swelling synthetic mica swells upon moisture adsorption to form a colloid or film, also exhibits ion-exchanging ability and thixotropy, and forms an inorganic complex with montmorillonite or other clay compounds. Amount of the synthetic mica to be added is generally 5 to 50% by weight to obtain a sufficient mechanical strength for the molded catalysts. Practically, an amount of 10 to 20% by weight suffices. Beside the swelling synthetic mica, other binders or modifiers may suitably be added to improve the operability at the molding process, for example, extrudability and thixotropy. For such purposes, silica, alumina, titania, zirconia, yttria, sericite, kaolinite, montmorillonite, and the like, are preferably used.

Though an amount of 5 to 50% by weight of a binder in the molded crystalline molecular sieve catalyst yields a sufficient mechanical strength, an amount exceeding 50% by weight causes no specific problem. An adding amount may be determined, accounting for the catalyst performances. The molded catalyst may preferably be prepared by adding a swelling synthetic mica, as binder, and water to a crystalline molecular sieve, and kneading the mixture, followed by extrusion, drying and calcination. The amount of water added before the kneading can not be defined beforehand, but it may be determined through observation of the film forming state when a mixture of a crystalline molecular sieve and water is kneaded on a glass plate. Kneading is preferably conducted under a superatmospheric pressure, and in a continuous way using a kneader from a view point of operability.

A main object of the drying step after the extrusion is to remove moisture. The step is conducted generally at a temperature of 80° C. to 150° C. for one to 10 hours, although conditions outside such ranges cause no problem. After the drying, the molded catalyst is arranged to the desired size, and then calcined normally in an oxidizing atmosphere, such as air. The temperature and period of time for the calcination vary depending upon the kind of molded catalysts, but generally they are 400° C. to 700° C. and 1 to 10 hours. The molded catalyst of the present invention has a sufficient mechanical strength even with a small amount of binder added.

The molded catalyst according to the present invention may be used for the production of methylamines through a reaction of methanol and ammonia, as well as the production of methylamines through a disproportionation reaction of monomethylamine.

The invention will more fully be described in reference to Examples and Comparative Examples, in which the reaction was conducted using a flowing reaction apparatus equipped with a material tank, material feeding pump, inert gas introducing means, reaction tube (13 Øinner diameter, 300 mm length, SUS 316L), sampling tank, back pressure bulb, etc. Six hours after the reaction reaches to the stationary condition, a sample of the product was recovered during 1 hour, and analyzed by gas chromatography to estimate the product distribution.

### EXAMPLE 1

A mixture of SAPO-34 (10 g) as the molecular sieve, swelling synthetic mica (ME-100, manufactured by CO-OP CHEMICAL Co. Ltd., 1.75 g), anatase-type titania (0.2 g) and water (10 g) was kneaded well, extruded using an injection cylinder, and then dried at 110° C. for 4 hours. After arranging the length, the product was calcined at 600° C. for 4 hours in an air stream. The resulting molded catalyst showed a press disruptive strength of as high as 19.0 N/mm. The molded matter was pulverized to give Catalyst 1 having a uniform 1 to 2 mm size, onto which a mixture of methanol and ammonia in 1:1 weight ratio was supplied at a time space velocity (GHSV) of 2500 h⁻¹. The catalyst activity for the production of methylamines after 6 hour reaction at a temperature of 320° C. under a pressure of 2 MPa was as follows:

| | |
|---|---|
| Methanol conversion: | 97.1% |
| Selectivity: | |
| monomethylamine | 33% by weight |
| dimethylamine | 63% by weight |
| trimethylamine | 4% by weight |

### Comparative Example 1

A molded catalyst was obtained in the same way as in Example 1, except that attapulgite was used in place of the swelling synthetic mica. The molded catalyst showed a press disruptive strength of 7.8 N/mm, which was at a usable level for catalyst filling, but there was a concern about collapse of the molded catalyst through pulverization. The results of the activity tests under the same conditions as in Example 1 are as follows:

| | |
|---|---|
| Methanol conversion: | 94.1% |
| Selectivity: | |
| monomethylamine | 33% by weight |
| dimethylamine | 54% by weight |
| trimethylamine | 13% by weight |

### Comparative Example 2

A molded catalyst was obtained in the same way as in Example 1, except that sepiolite was used in place of the swelling synthetic mica, with a press disruptive strength of 7.0 N/mm. Results of the activity tests under the same conditions as in Example 1 are as follows:

| | |
|---|---|
| Methanol conversion: | 91.1% |
| Selectivity: | |
| monomethylamine | 34% by weight |
| dimethylamine | 56% by weight |
| trimethylamine | 10% by weight |

### Comparative Example 3

A molded catalyst was obtained in the same way as in Example 1, except that alumina was used in 15% by weight amount as binder, with a press disruptive strength of 5.7 N/mm.

### EXAMPLE 2

A molded catalyst was obtained in the same way as in Example 1, except that 25% by weight of the swelling synthetic mica was used. The catalyst showed a press disruptive strength of 28.0 N/mm. Activity tests were conducted under the same conditions as in Example 1, with the results as follows:

| | |
|---|---|
| Methanol conversion: | 96.1% |
| Selectivity: | |
| monomethylamine | 33% by weight |
| dimethylamine | 55% by weight |
| trimethylamine | 12% by weight |

### EXAMPLE 3

A molded catalyst was obtained in the same way as in Example 1, except that 10% by weight of the swelling synthetic mica was used. The catalyst showed a press disruptive strength of 8.3 N/mm.

### Comparative Example 4

A molded catalyst was obtained in the same way as in Comparative Example 1, except that 10% by weight of attapulgite was used, with a press disruptive strength of 4.9 N/mm.

### Comparative Example 5

A molded catalyst was obtained in the same way as in Comparative Example 1, except that 10% by weight of sepiolite was used, with a press disruptive strength of 3.2 N/mm.

### EXAMPLE 4

A molded catalyst was obtained in the same way as in Example 1, except that mordenite was used in place of SAPO-34. The catalyst showed a press disruptive strength of 16.0 N/mm.

### EXAMPLES 5 THROUGH 16

Molded catalysts were obtained in the same way as in Example 4 using chabazite, erionite, ferrierite, ZSM-5, zeolite A, zeolite Y, zeolite β, SAPO-5, SAPO-11, SAPO-18, SAPO-47 or MCM-22, respectively, in place of the mordenite. Press disruptive strengths of the resulting molded catalyst are as shown in Table 1.

TABLE 1

| | | | | Disrup- | MeOH | | | |
|---|---|---|---|---|---|---|---|---|
| | | | Amount | tive | conver- | Selectivity | | |
| Exam- | | | added | strength | sion | (% by wt.) | | |
| ples | Catalysts | Binders | % by wt. | N/mm | % | m-MA | d-Ma | t-MA |
| Ex. 1 | SAPO-34 | ME-100 | 15 | 19.0 | 97.1 | 33 | 63 | 4 |
| Ex. 2 | SAPO-34 | ME-100 | 25 | 28.0 | 96.3 | 33 | 55 | 12 |
| Ex. 3 | SAPO-34 | ME-100 | 10 | 8.3 | 98.1 | 33 | 64 | 3 |
| Comp. Ex. 1 | SAPQ-34 | attapulgite | 15 | 7.8 | 94.1 | 33 | 54 | 13 |
| Comp. Ex. 2 | SAPO-34 | sepiolite | 15 | 7.0 | 91.1 | 34 | 56 | 10 |
| Comp. Ex. 3 | SAPO-34 | alumina | 15 | 5.7 | 96.4 | 32 | 51 | 17 |
| Comp. Ex. 4 | SAPO-34 | attapulgite | 10 | 4.9 | | | | |
| Comp. Ex. 5 | SAPO-34 | sepiolite | 10 | 3.2 | | | | |
| Ex. 4 | mordenite | ME-100 | 15 | 16.0 | | | | |
| Ex. 5 | chabazite | ME-100 | 15 | 15.2 | | | | |
| Ex. 6 | erionite | ME-100 | 15 | 14.8 | | | | |
| Ex. 7 | ferrierite | ME-100 | 15 | 17.3 | | | | |
| Ex. 8 | ZSM-5 | ME-100 | 15 | 15.0 | | | | |
| Ex. 9 | zeolite A | ME-100 | 15 | 12.8 | | | | |
| Ex. 10 | zeolite β | ME-100 | 15 | 16.3 | | | | |
| Ex. 11 | zeolite Y | ME-100 | 15 | 13.2 | | | | |
| Ex. 12 | SAPO-5 | ME-100 | 15 | 14.5 | | | | |
| Ex. 13 | SAPO-11 | ME-100 | 15 | 16.8 | | | | |
| Ex. 14 | SAPO-18 | ME-100 | 15 | 11.9 | | | | |
| Ex. 15 | SAPO-47 | ME-100 | 15 | 17.4 | | | | |
| Ex. 16 | MCM-22 | ME-100 | 15 | 12.7 | | | | |

ME-100: swelling synthetic fluorine mica (CO-OP CHEMICAL Co. Ltd.)
m-MA: monomethylamine
d-MA: dimethylamine
t-MA: trimethylamine
Reaction conditions: temperature 320° C., pressure 2 MPa and GHSV 2500 h⁻¹

The results from the examples of the present invention, as well as from the comparative examples, are shown in Table 1. They show that the molded catalysts according to the present invention exhibit sufficient mechanical strengths, even with a small amount of binder added, thus, providing a significant and useful technology.

1. A molded catalyst which comprises a crystalline aluminosilicate molecular sieve or crystalline silicoaluminophosphate molecular sieve, containing swelling synthetic mica, as binder.

2. A molded catalyst according to claim 1, which contains silica, alumina, titania, zirconia, yttria, sericite, kaolinite or montmorillonite.

3. A molded catalyst according to claim 1, wherein the crystalline aluminosilicate molecular sieve or the crystalline silicoaluminophosphate molecular sieve contains Li, Na, Be, Mg, Ca, Sr, Y, Ti, Zr, V, Nb, Cr, Mn, Fe, Ru, Co, Rh, Ir, Ni, Pd, Pt, Cu, Zn, B, Ga, In, Ge or Sn.

4. A molded catalyst according to claim 1, wherein the crystalline aluminosilicate molecular sieve is mordenite, chabazite, erionite, ferrierite, faujasite, levyne, ZSM-5, zeolite A, zeolite β, zeolite Y, FU-1, Rho, ZK-5, RUB-3, RUB-13, NU-3, NU-4, NU-5, NU-10, NU-13, NU-23 or MCM-22.

5. A molded catalyst according to claim 1, wherein the crystalline silicoaluminophosphate molecular sieve is SAPO-5, SAPO-11, SAPO-17, SAPO-18, SAPO-26, SAPO-31, SAPO-33, SAPO-34, SAPO-35, SAPO-42, SAPO-43, SAPO-44, SAPO-47 or SAPO-56:

6. A molded catalyst according to claim 1, wherein the crystalline molecular sieve is mordenite containing Ti, Y or Zr, or SAPO-34 containing Ti, Y or Zr, being used for producing methylamines.

7. A molded catalyst according to claim 1, wherein the amount of the binder in the molded catalyst is 5 to 50 % by weight on the basis of catalyst.

8. A process for producing methylamines, which comprises allowing methanol to react with ammonia in the presence of a molded catalyst as claimed in claim 1.

9. A process for producing methylamines, which comprises subjecting monomethylamine to a disproportionation reaction in the presence of a molded catalyst as claimed in claim 1.
